(19) United States
(12) Patent Application Publication
(10) Pub. No.: US 2020/0113792 A1
(43) Pub. Date: Apr. 16, 2020

(54) Stabilized Hydrogen Peroxide Composition

(71) Applicant: Cavex Holland B.V., Haarlem (NL)

(72) Inventors: Matthew Spaid, Amsterdam (NL); Richard Woortman, Velserbroek (NL); Peter Helversteijn, Hillegom (NL); Johannes Maarten den Boer, Heiloo (NL)

(73) Assignee: Cavex Holland B.V., Haarlem (NL)

(21) Appl. No.: 16/597,070

(22) Filed: Oct. 9, 2019

(30) Foreign Application Priority Data
Oct. 11, 2018 (EP) 18 199 772.7

(51) Int. Cl. A61K 8/22; A61K 8/81; A61K 8/90; A61Q 11/00; A61K 8/24

(52) U.S. Cl. CPC A61K 8/22; A61K 8/8182; A61K 8/90; A61K 2800/51; A61K 8/24; A61K 2800/592; A61K 2800/522; A61Q 11/00

(57) ABSTRACT

A stabilized aqueous hydrogen peroxide composition comprising an encapsulating or matrix-forming polymer is disclosed. The composition comprises 2 to 30 wt. % of hydrogen peroxide, water, 1 to 10 wt. % poly(ethylene oxide)-poly(propylene oxide) block copolymer, 0.01 to 10 wt. % of a salt of a polymer or co-polymer based on at least a taurate, taurate salt, 2-alkyltaurate, 2-alkyltaurate salt, 2,2-dialkyltaurate, 2,2-dialkyltaurate salt or a mixture of a plurality of these taurates, and at least an acrylic acid, acrylic acid derivative, or comprising co-polymers of acrylic acid, and at least one ethylenically unsaturated monomer or a mixture thereof, and 30 to 95 wt. % poly(ethylene oxide) homopolymer, 0.01 to 5 wt. % stabilizer system comprising 0.1 to 2 wt. % and 0.01 to 1 wt. % of a chelating agent or a mixture of chelating agents, wherein the composition comprises in total 100 wt. %.

This application claims priority of European Patent Application No. 18 199 772.7, filed Oct. 11, 2018, the contents of which are hereby incorporated herein by reference.

Disclosed is a stabilized aqueous hydrogen peroxide composition comprising polymers with N containing groups for encapsulating a mixture comprising at least hydrogen peroxide, water and poly(ethylene oxide) homopolymer, or for forming a matrix comprising a mixture of at least hydrogen peroxide, water and poly(ethylene oxide) homopolymer.

The composition comprising 2 wt. % to 30 wt. % of hydrogen peroxide, an amount of water, 1 wt. % to 10 wt. % poly(ethylene oxide)-poly(propylene oxide) block copolymer, 0.01 wt. % to 10 wt. % of a salt of a polymer or co-polymer comprising polymers based on at least a taurate, taurate salt, 2-alkyltaurate, 2-alkyltaurate salt, 2,2-dialkyltaurate, 2,2-dialkyltaurate salt or a mixture of at least two of the aforementioned taurates, and at least an acrylic acid, acrylic acid derivative, or comprising co-polymers of acrylic acid, in particular acryolyl-2-alkyltaurate, acryolyl-2,2-dialkyltaurate, acryolyl-2-alkyltaurate salt or acryolyl-2,2-dialkyltaurate salt or a mixture of at least two of the aforementioned taurates, and at least one ethylenically unsaturated monomer or a mixture thereof, wherein the alkyl is selected from methyl, ethyl, n-propyl, iso-propyl, n-butyl, iso-butyl, tert-butyl groups, and wherein the salt is selected from ammonium, alkali, earth alkali or zinc salts of these polymers or co-polymers, in particular ammonium or alkali salt of a copolymer of acryloyldimethyltaurate and vinylpyrrolidone, and 30 wt. % to 95 wt. % poly(ethylene oxide) homopolymer, 0.01 wt. % to 5 wt. % stabilizer system comprising 0.1 to 2 wt. % and 0.01 wt. % to 1 wt. % of a chelating agent or a mixture of chelating agents, wherein the composition comprises in total 100 wt. %.

Many bleaching compositions are known. U.S. Pat. No. 7,341,636B2 discloses an alkaline hydrogen peroxide composition comprising aromatic chelating agents and a pH above 7.0.

In general, hydrogen peroxide is acceptable for use in bleaching or whitening compositions with regard to the decomposition products water and oxygen. However, the unintended decomposition of hydrogen peroxide due to contaminations of substances with traces of metal ions makes the utilization of such applications very difficult. For acidic compositions with a pH less than 5.0 stabilizers such as sodium stannate, phosphorus compounds or other chelating agents are known.

Object of the invention was to provide a composition with a less acidic pH, in particular in the range of above 5.0 and less than 7.0, wherein the composition shall be stable towards decomposition of hydrogen peroxide, in particular at room temperature. Further the composition should release the peroxide quickly. In addition, the composition should have a variable viscosity to enable the preparation of compositions to be applied by e.g. syringe, pen, bite splint or single-dose. One particular object was to provide a composition that is stable at room temperature. Moreover, it was an object of the invention to provide a composition which remains on the tooth surface with a high penetration potential.

According to the invention a composition is disclosed, wherein the composition comprises
i 2 wt. % to 30 wt. % of hydrogen peroxide
ii water, in particular 2 wt. % to 30 wt. % water,
iii 1 wt. % to 10 wt. % poly(ethylene oxide)-poly(propylene oxide) block copolymer, in particular the copolymer is a mixture of these copolymers and is selected from poly(ethylene oxide)-poly(propylene oxide)-poly(ethylene oxide) block copolymers and a mixture comprising poly(ethylene oxide)-poly(propylene oxide)-poly(ethylene oxide) block copolymers, preferred with terminal hydroxyl groups,
iv 0.01 wt. % to 10 wt. % of a salt of a polymer or co-polymer comprising polymers based on at least a taurate, taurate salt, 2-alkyltaurate, 2-alkyltaurate salt, 2,2-dialkyltaurate, 2,2-dialkyltaurate salt or a mixture of at least two of the aforementioned taurates, and at least an acrylic acid, acrylic acid derivative, or comprising co-polymers of acrylic acid, in particular acryolyl-2-alkyltaurate, acryolyl-2,2-dialkyltaurate, acryolyl-2-alkyltaurate salt or acryolyl-2,2-dialkyltaurate salt or a mixture of at least two of the aforementioned taurates, and at least one ethylenically unsaturated monomer or a mixture thereof, wherein the alkyl is selected from methyl, ethyl, n-propyl, iso-propyl, n-butyl, iso-butyl, tert-butyl groups, and wherein the salt is selected from ammonium, alkali, earth alkali or zinc salts of these polymers or co-polymers,
v 30 wt. % to 95 wt. % poly(ethylene oxide) homopolymer, in particular 35 wt. % to 85 wt. of poly(ethylene oxide) homopolymer,
vi 0.01 wt. % to 5 wt. % stabilizer system, in particular 0.1 to 2 wt. %,
vii 0.01 wt. % to 1 wt. % of a chelating agent or a mixture of chelating agents, wherein the composition comprises in total 100 wt. %.

A salt of a polymer comprising polymers based on at least a taurate, taurate salt, 2-alkyltaurate, 2-alkyltaurate salt, 2,2-dialkyltaurate, 2,2-dialkyltaurate salt or a mixture of at least two of the aforementioned taurates, and at least an acrylic acid, acrylic acid derivative can be selected from polymers and mixtures of polymers based on acryolyl-2-alkyltaurate, acryolyl-2,2-dialkyltaurate, acryolyl-2-alkyltaurate salt or acryolyl-2,2-dialkyltaurate salt or a mixture of at least two of the aforementioned taurates.

The composition can be used as an oral care application, in particular as a tooth bleaching composition/tooth whitening composition. The viscosity of the composition may be adapted to easily enable the application via syringe, pen or single-dose application.

A preferred composition has reversible temperature-sensitive properties; in particular, the composition is a thermo-sensitive gel. Further, in a preferred embodiment the composition of the present invention possesses a sole-state at room temperature and a gel-state at a physiological temperature, the latter being in the range of about 35 to 38° C. or around 37° C.

In a preferred embodiment, compound iv the polymer is based on a taurate, taurate salt, 2-alkyltaurate, 2-alkyltaurate salt, 2,2-dialkyltaurate, 2,2-dialkyltaurate salt or a mixture of at least two of the aforementioned taurates and acrylic acid, acrylic acid derivative and/or co-polymer is based on acryolyl-2-alkyltaurate, acryolyl-2,2-dialkyltaurate, acryolyl-2-alkyltaurate salt or acryolyl-2,2-dialkyltaurate salt and vinylpyrrolidone, wherein the alkyl is selected from methyl, and wherein the salt is selected form ammonium, alkali, earth alkali or zinc salts of these polymers or co-polymers. In a more preferred embodiment the compound iv polymer is a co-polymer of an ammonium or alkali salt of acryloyldimethyltaurate and ethylene, acryloyldimethyltaurate and styrene or acryloyldimethyltaurate and vinylpyrrolidone, in particular an ammonium salt, most preferred is an ammonium salt of copolymer of acryloyldimethyltaurate and vinylpyrrolidone (Acrylamido methyl propane sulfonic acid polymer).

A preferred compound iv of the above mentioned polymer or co-polymer has a molecular weight in the range from 10000 to 450000 g/mol, in particular from 100000 g/mol to 350000 g/mol, more preferred from 150000 to 275000 g/mol. Most preferred is a molecular weight in the range of 225000 g/mol+/−50000 g/mol. Further, it is preferred when the compound iv the polymer or co-polymer has a viscosity in the range from 45000 to 67000 mPas (1 Gew.-% in dist. H₂O).

Compound iv may be based on polymers of acrylic acid derivatives and/or co-polymers of acrylic acid, in particular acryolyl-2,2-dialkyltaurate or acryolyl-2,2-dialkyltaurate salt, and at least one ethylenically unsaturated monomer e.g. olefinic monomer, e.g. vinylpyrrolidone or fatty acids, as mentioned below. In addition, in some cases it may be preferred to use cross-linked polymers or co-polymers.

Alternatively preferred compounds iv comprise co-polymers of acryolyl-2-alkyltaurate, acryolyl-2,2-dialkyltaurate, acryolyl-2-alkyltaurate salt, acryolyl-2,2-dialkyltaurate salt or a mixture of at least two of the aforementioned taurates and ethylenically unsaturated monomer, preferred are co-polymers or mixtures of co-polymers based on acryloyldimethyltaurate and least one ethylenically unsaturated monomer selected from vinylpyrrolidone, styrene and vinyl-polyethylene oxide.

Particular preferred compounds iv comprise: Acrylamide/Sodium Acryloyldimethyltaurate Copolymer, Acrylamide/Sodium Acryloyldimethyltaurate/Acrylic Acid Copolymer, Ammonium Acryloyldimethyltaurate/Beheneth-25 Methacrylate Crosspolymer. Ammonium Acryloyldimethyltaurate/Carboxyethyl Acrylate Crosspolymer, Ammonium Acryloyldimethyl-taurate/Laureth-7 Methacrylate Copolymer, Ammonium Acryloyldimethyltaurate/Steareth-25 Methacrylate Crosspolymer, Ammonium Acryloyldimethyltaurate/Steareth-8 Methacrylate Copolymer, Ammonium Acryloyldimethyltaurate/Vinyl Formamide Copolymer, Ammonium Acryloyldimethyltaurate/VP Copolymer, Ammonium Polyacryloyldimethyl Taurate, Dimethylacrylamide/Sodium Acryloyldimethyltaurate Crosspolymer, HEA/Sodium Acryloyldimethyltaurate/Steareth-20 Methacrylate Copolymer, Hydroxyethyl Acrylate/Sodium Acryloyldimethyl Taurate Copolymer, Polyacryloyldimethyltaurate Polyoxymethylene Melamine, Sodium Acrylate/Acryloyldim ethyltaurate/Dimethylacrylamide Crosspolymer, Sodium Acrylate/Sodium Acryloyldimethyl Taurate Copolymer, Sodium Acrylate/Sodium Acryloyldimethyl Taurate/Acrylamide Copolymer, Sodium Acryloyl Dimethyl Taurate/PEG-8 Diacrylate Crosspolymer, Sodium Acryloyldimethyl Taurate/Acrylamide/VP Copolymer, Sodium Acryloyldimethyltaurate/Methacrylamidolauric Acid Copolymer and/or Sodium Acryloyldimethyltaurate/VP Crosspolymer, Sodium Polyacryloyldimethyl Taurate as well as mixtures comprising at least two of the aforementioned polymers or co-polymers.

The composition according to the invention comprises in a preferred embodiment from 0.5 wt. % to 6 wt. %, more preferred 0.5 wt. % to 5 wt. %, of a salt, in particular of an ammonium or alkali salt, of a copolymer of acryloyldimethyltaurate and vinylpyrrolidone, preferred is an ammonium salt of a copolymer of acryloyldimethyltaurate and vinylpyrrolidone. The ethylenically unsaturated monomer may be selected from N-containing ethylenically unsaturated monomers, such as in particular vinylpyrrolidone, vinylpyrrolidone derivates. Alternatively the ethylenically unsaturated monomer may be selected from ethen, propen, styrene, buten, butadiene or the like.

Preferred co-polymers comprise co-polymers of acrylic acid and vinylpyrrolidone, co-polymers of methacrylic acid and vinylpyrrolidone or a mixture thereof.

The used poly(ethylene oxide)-poly(propylene oxide) block copolymer or a mixture of these co-polymers is selected from a co-polymer having poly(ethylene oxide) blocks and poly(propylene oxide) blocks, wherein in particular the co-polymer possesses terminal poly(ethylene oxide) blocks with terminal hydroxyl groups and poly(propylene oxide) blocks in between. The co-polymer comprising poly(ethylene oxide)-poly(propylene oxide)-poly(ethylene oxide) block, and in particular with terminal hydroxyl groups forms micelles and/or functions as emulgator and controlling agent to adjust the viscosity of the composition.

The used poly(ethylene oxide)-poly(propylene oxide) block copolymer, in particular poly(ethylene oxide)-poly(propylene oxide)-poly(ethylene oxide) block forms micelles and/or functions as emulgator and controlling agent to adjust the viscosity of the composition.

Typically molecular weights of the poly(ethylene oxide)-poly(propylene oxide) block copolymer, in particular poly(ethylene oxide)-poly(propylene oxide)-poly(ethylene oxide) block, may range from 1000 g/mol to 15000 g/mol, preferably from 2000 g/mol to 15000 g/mol, most preferred is from 1000 to 5000 g/mol. Particular a preferred poly(ethylene oxide)-poly(propylene oxide)-poly(ethylene oxide) block co-polymer is selected from poly(ethylene oxide)-poly(propylene oxide)-poly(ethylene oxide) block copolymers and a mixture comprising poly(ethylene oxide)-poly(propylene oxide)-poly(ethylene oxide) block copolymers with terminal hydroxyl groups, preferably with a molecular weight in the range form from 1000 g/mol to 15000 g/mol, preferably from 2000 g/mol to 15000 g/mol, most preferred is from 1000 to 5000 g/mol. A particular preferred poly(ethylene oxide)-poly(propylene oxide)-poly(ethylene oxide) block co-polymer is selected from a mixture comprising poly(ethylene oxide)-poly(propylene oxide)-poly(ethylene oxide) block copolymers with terminal hydroxyl groups with a molecular weight in the range form from 1.500 g/mol to 10.000 g/mol, preferably from 2000 g/mol to 4000 g/mol.

The poly(ethylene oxide)-poly(propylene oxide) block copolymer is used as a thermo-reversible gelling agent. In particular the poly(ethylene oxide)-poly(propylene oxide) block copolymer and the poly(ethylene oxide) homopolymer are used in combination to provide a thermo-adapting gel composition, in particular a thermo-reversible thermoplastic gel. In particular the poly(ethylene oxide)-poly(propylene oxide) block copolymer and the poly(ethylene oxide) homopolymer mixture swells in the mouth at the temperature of a human being.

The poly(ethylene oxide) homopolymer forms a mixture with hydrogen peroxide and water. Preferred poly(ethylene oxide) homopolymers have a low molar mass of below 10.000 g/mol. Further a preferred poly(ethylene oxide) homopolymer comprises terminal hydroxyl groups. A particular preferred poly(ethylene oxide) homopolymer has a molar mass of 100 to 5.000 g/mol, more preferred form 100 to below 500 g/mol. The most preferred poly(ethylene oxide) homopolymer has a molar mass of 150 to 250 g/mol, e.g. PEG 200 (molar mass 190-210 g/mol).

A particular preferred composition comprises
i 5 wt. % to 15 wt. % of hydrogen peroxide
ii 5 wt. % to 15 wt. % of water
iii 2 wt. % to 10 wt. % poly(ethylene oxide)-poly(propylene oxide) block copolymer
iv 0.01 wt. % to 10 wt. % salt of a polymer based on a taurate, taurate salt, 2-alkyltaurate, 2-alkyltaurate salt, 2,2-dialkyltaurate, 2,2-dialkyltaurate salt or a mixture of at least two of the aforementioned taurates and an acrylic acid, acrylic acid derivative and/or co-polymer of acrylic acid, in particular acryolyl-2,2-dialkyltaurate or acryolyl-2,2-dialkyltaurate salt, and at least one ethylenically unsaturated monomer, wherein the monomer is selected from vinylpyrrolidone, vinylpyrrolidone derivatives, wherein the alkyl is selected from methyl, ethyl, n-propyl, iso-propyl, n-butyl, iso-butyl, tert-butyl groups, and wherein the salt is selected from ammonium, alkali, earth alkali or zinc salts of these polymers or co-polymers,
v 60 wt. % to 95 wt. % poly(ethylene oxide) homopolymer, in particular with terminal hydroxy groups, particularly preferred 65 wt. % to 85 wt. % of poly(ethylene oxide) homopolymer,
vi 0.01 wt. % to 5 wt. % stabilizer system, in particular 0.1 to 2 wt. %,
vii 0.01 wt. % to 1 wt. % of a chelating agent or a mixture of chelating agents, wherein the composition comprises in total 100 wt. %. In a preferred embodiment the content of i hydrogen peroxide and ii water is equal.

A further particular preferred composition comprises
i 5 wt. % to 15 wt. % of hydrogen peroxide
ii 5 wt. % to 15 wt. % of water
iii 2 wt. % to 10 wt. % poly(ethylene oxide)-poly(propylene oxide) block copolymer
iv 0.01 wt. % to 10 wt. %, preferred 0.1 to 5 wt.-% salt of a polymer based on a taurate, taurate salt, 2-alkyltaurate, 2-alkyltaurate salt, 2,2-dialkyltaurate, 2,2-dialkyltaurate salt or a mixture of at least two of the aforementioned taurates and an acrylic acid, acrylic acid derivative and/or co-polymer of acrylic acid, in particular acryolyl-2,2-dialkyltaurate or acryolyl-2,2-dialkyltaurate salt, and at least one ethylenically unsaturated monomer, wherein the monomer is selected from vinylpyrrolidone, vinylpyrrolidone derivatives, wherein the alkyl is selected from methyl, ethyl, n-propyl, iso-propyl, n-butyl, iso-butyl, tert-butyl groups, and wherein the salt is selected from ammonium, alkali, earth alkali or zinc salts of these polymers or co-polymers, v 30 wt. % to 55 wt. % poly(ethylene oxide) homopolymer, in particular with terminal hydroxy groups, particularly preferred 35 wt. % to 50 wt. % of poly(ethylene oxide) homopolymer,
vi 0.01 wt. % to 5 wt. % stabilizer system, in particular 0.1 to 2 wt. %,
vii 0.01 wt. % to 1 wt. % of a chelating agent or a mixture of chelating agents, wherein the composition comprises in total 100 wt. %. In a preferred embodiment the content of i hydrogen peroxide and ii water is equal.

An alternative preferred composition comprises
i 15 wt. % to 30 wt. % of hydrogen peroxide
ii 15 wt. % to 30 wt. % of water,
iii 2 wt. % to 10 wt. % poly(ethylene oxide)-poly(propylene oxide) block copolymer
iv 0.01 wt. % to 10 wt. % salt of a co-polymer or a mixture of co-polymers of acryolyl-2,2-dialkyltaurate or acryolyl-2,2-dialkyltaurate salt and at least one ethylenically unsaturated monomer, wherein the monomer is selected from vinylpyrrolidone, vinylpyrrolidone derivatives, wherein the alkyl is selected from methyl, ethyl, n-propyl, iso-propyl, n-butyl, iso-butyl, tert-butyl groups, and wherein the salt is selected from ammonium, alkali, earth alkali or zinc salts of these polymers or co-polymers,
v 30 wt. % to 60 wt. % poly(ethylene oxide) homopolymer, in particular with terminal hydroxy groups, particularly preferred 35 wt. % to 50 wt. % of poly(ethylene oxide) homopolymer,
vi 0.01 wt. % to 5 wt. % stabilizer system, in particular 0.1 to 2 wt. %,
vii 0.01 wt. % to 1 wt. % of a chelating agent or a mixture of chelating agents, wherein the composition comprises in total 100 wt. %. In a preferred embodiment the content of i hydrogen peroxide and ii water is equal.

In one embodiment the composition may comprise a weight content (wt. %) of hydrogen peroxide and water in the range from 2:1 to 1:2, in particular 1:1+/−10 wt. %.

As compositions comprising aqueous hydrogen peroxides are typically acidic, it may be necessary to add a base to the composition, such as aqueous potassium hydroxide or aqueous sodium hydroxide until the desired pH is attained. Aqueous potassium hydroxide is preferred. The base or alkali should be free from metal ions that would catalyse the decomposition of the hydrogen peroxide, such as ferrous ions, ferric ions, cupric ions, cuprous ions, manganous ions, and similar transition metal ions. The base or alkali should also be free from both organic and inorganic materials that would react with the hydrogen peroxide. The stabilized composition comprising aqueous hydrogen peroxide typically comprises about 1 wt. % to about 30 wt. %, typically about 4 wt. % to about 20 wt. %, more typically about 4 wt. % to about 12 wt. %, even more typically about 5 wt. % to about 10 wt. %, of hydrogen peroxide. Further it is preferred that the components i to v, in particular the hydrogen peroxide and all polymers, each comprise less than 10 ppm-wt. % metal ions.

In one embodiment, component vii, the chelating agent or the mixture of chelating agents, is selected from ethylenediaminetetraacetic acid, alkali and/or alkali earth salt of ethylene-diaminetetraacetic acid (EDTA), aromatic chelating agents and a mixture thereof, in particular alkali and/or alkali earth salt of ethylenediaminetetraacetic acid (EDTA), particularly preferred are potassium and/or sodium EDTA. Preferred aromatic chelating agents may be selected from salts of salicylic acid, substituted salicyclic acid, 6-hydroxy-piconilic acid, substituted 6-hydroxy-picolinic acid, 8-hydroxy-quinoline, and substituted 8-hydroxy-quinolines.

In a further embodiment, the composition comprising vi: a stabilizer system, wherein the stabilizer system is selected from

a) phosphorus containing stabilizer or mixture of phosphorus containing stabilizers selected from the group consisting of pyrophosphate, in particular diethylene triamine penta(methylene phosphonic acid), hexamethylene diamine tetra(methylene phosphonic acid), bis hexamethylene triamine pentamethylene phosphonic acid and etidronic acid, and/or
b) stannate stabilizer, and/or
c) alkali hydroxide, base and/or buffers, preferred are potassium hydroxide and/or sodium hydroxide. A particular preferred vi stabilizer system is selected from a) etidronic acid (1-hydroxyethane-1,1-diphosphonic acid) and b) stannate stabilizer, in particular sodium stannate trihydrate.

A preferred poly(ethylene oxide)-poly(propylene oxide) block copolymer, in particular a poly(ethylene oxide)-poly(propylene oxide)-poly(ethylene oxide) block is selected from poly(ethylene oxide)-poly(propylene oxide)-poly(ethylene oxide) block copolymers, in particular with terminal hydroxyl groups, with a medium molecular weight of 9840 to 14600 Dalton (Dalton D=unit for the relative molecular mass). The hydroxy value of the block copolymer may be from 44.0 to 55.0 (KOH/g % w/w). In addition the pH of the block copolymer may be in the range of 5.0 to 7.5 (2.5% in water).

The pH of a preferred composition is higher than 5.0, in particular the pH is in the range of 5.0 to 7.5, and particular preferred is a pH in the range of 5.1 to 6.5.

The viscosity of the composition may vary from 1 to 1000 Pa·s, preferred is a range from 5 to 200 Pa·s (ISO28399). At 25° C. the viscosity remains stable over (at least) 12 months remained in the range of 10 to 100 Pa·s, in particular in the range of 20 to 45 Pa·s.

A further embodiment of the invention is the use of a salt of a polymer or co-polymer comprising at least one N-group based on a taurate or taurate derivate for encapsulation of a mixture comprising at least hydrogen peroxide, water and poly(ethylene oxide) homopolymer, or for forming a matrix comprising a mixture of at least hydrogen peroxide, water and poly(ethylene oxide) homopolymer.

Acrylic acid derivatives in the salt of a polymer or co-polymer are particular based on at least a taurate, taurate salt, 2-alkyltaurate, 2-alkyltaurate salt, 2,2-dialkyltaurate, 2,2-dialkyltaurate salt or a mixture of at least two of the aforementioned taurates and at least an acrylic acid derivative that may comprise methacrylic acid.

It will be apparent to those skilled in the art, that in aqueous neutral, acidic or basic solution one or more of the above mentioned components may be present as its corresponding anion or anions, or as an equilibrium mixture of the component and its corresponding anion or anions. Anions of these components and mixtures of these components and their corresponding anions are included in the definition of each of these components and are within the scope of the claims.

The advantageous properties of this invention are shown by the following examples, which illustrate the invention without limiting the invention to these examples.

### BRIEF DESCRIPTION OF THE DRAWING

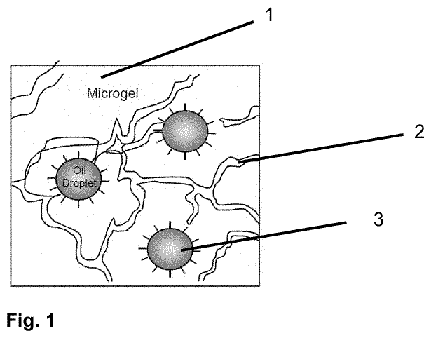

A schematically illustration of the microgel 1 and the encapsulation by the ammonium salt of the co-polymer of acrylic acid and vinylpyrrolidone 2 of the micelles 3 forming the oil droplets is shown in FIG. 1.

TABLE 1
Examples 1 to 3

| | belongs | example 1 | example 2 | example 3 |
|---|---|---|---|---|
| compound | to | wt. % | wt. % | wt. % |
| PEG 200 | v | 40 | 72 | 78 |
| H₂O₂ | i | 25 | 9 | 6 |
| water | ii | 25 | 9 | 6 |
| Poloxamer | iii | 5 | 5 | 5 |
| Aristoflex | iv | 1 | 1 | 1 |
| KOH | vi | <1 | <1 | <1 |
| Etidronic acid | vi | <1 | <1 | <1 |
| EDTA (potassium) | vii | <1 | <1 | <1 |
| Sodium stannate | vi | <1 | <1 | <1 |
| trihydrate | | | | |

TABLE 2
Examples 4 to 6

| | belongs | example 4 | example 5 | example 6 |
|---|---|---|---|---|
| compound | to | wt. % | wt. % | wt. % |
| PEG 200 | v | 40 | 72 | 78 |
| H₂O₂ | i | 25 | 9 | 6 |
| water | ii | 25 | 9 | 6 |
| Poloxamer | iii | 5 | 5 | 5 |
| Aristoflex | iv | 4 | 4 | 4 |
| KOH | vi | <1 | <1 | <1 |
| Etidronic acid | vi | <1 | <1 | <1 |
| EDTA (potassium) | vii | <1 | <1 | <1 |
| Sodium stannate | vi | <1 | <1 | <1 |
| trihydrate | | | | |

The viscosity of these compositions is in the range of 10 to 100 Pa·s (ISO28399).

12 month stability with measurement of pH, viscosity and peroxide concentration:

pH (acidity): pH meter (Hanna Instruments pH210) with including sensor, glass beaker 50 ml.

Beaker was filed with gel and the sensor placed in the gel, wherein the gel covers the sensor. After 5 minutes the pH was collected.

Peroxide concentration iodometric.determination of the peroxide concentration in a bleach gel; Ingredients necessary: Potassium iodide ACS reagent; Aldrich 221945, Potassium iodide solution 10%, sulphuric acid 95-97% Aldrich ACS reagent; 320501, 1 M (2N) sulphuric acid, Soluble starch 1%, 0.1000±0.001 M Sodium thiosulphate; Fluka, analytical; product number 35245, calcium carbonate ACS reagent; Aldrich 256501,

Equipment: Erlenmeyer 200 ml, cylinder glass 100 ml and 25 ml, Magnet, Burette; Class A.

Preparation of the fresh potassium iodide: Glass Beaker 400 ml tare, (3 times rinse with demineralized water), Add 25 g±0.1 g potassium iodide, demineralized water till 250 g, Store in amber colored bottle.

Preparation of the sulphuric acid solution: Glass Beaker 1000 ml tare, 3 times rinse with demineralized water, Add 400 g demineralized water, 102.2 g sulphuric acid 95-97% and stir, Bottle 1 liter tare, sulphuric solution from beaker in the bottle, fill with demineralized water to 1061 g.

Preparation of the starch solution: Prepare fresh maximum 33 days, Glass beaker 150 ml tarr, Add 60 g demineralized water and heat, Prepare a paste of 1 g soluble starch and 3 ml demineralized water, Bring paste in 60 ml hot water, Fill with demineralized water to 102 g and warm till it boils, Leave for 1 minute, cover with watch glass, cool down and add 2 g potassium chloride, store refrigerated.

Working instructions: Remove peroxide sample several hours before testing from refrigerator, Weigh in a 200 ml Erlenmeyer 0.9 g of the gel, Add 25 ml demineralized water, Add 75 ml 2 N sulphuric acid, Add 15 mg Calcium carbonate and stir till completely dissolved, Add slowly 7.5 ml 10% potassium iodide, Make homogeneous, Titrate with 0.1000±0.0001 M sodium thiosulfate, When more than 20% is expected add fast 25 ml sodium thiosulfate, Add 1 ml starch solution, Do a blank sample using 1.25 ml demineralized water, Calculate peroxide as being carbamide peroxide.

Calculations:

H₂O₂+2 H⁺+2I⁻I₂+2 H₂O

2 S₂O₃²⁻+I₂→S₄O₆²—=2 I⁻

1 mol sodium thiosulfate 1/2 mol I₂ ˜1/2 mol H₂O₂ ˜17.01 g/mol H₂O₂ →1 ml 1 M sodium thiosulfate is 17.01 mg hydrogen peroxide.

$$Percentage {H}_{2} {O}_{2} = \frac{V \times M \times 17.01 \times 100}{weigh in mg}$$

Molmass carbamide peroxide is 94.07 g/mol.

→1 ml 1 M sodium thiosulfate is 47.035 mg carbamide peroxide.

$$Percentage carbamide peroxide = \frac{V \times M \times 47.035 \times 100}{Weigh in mg} = \frac{V \times 0.1000 \times 47.035 \times 100}{weigh in mg}$$

V=used in milliliter.

M=mol thiosulfate solution.

Viscosity (Haake Rheostress 1): Measured at 19.4° C., Open valve pressure air. Pressure 2.4 bar and not above 2.5 bar, Place 35 mm spindle in the device

TABLE 3
example 7

| | belongs | example 7 |
|---|---|---|
| compound | to | wt. % |
| PEG 200 | v | 40-45 |
| H₂O₂ | i | 9-10 |
| water | ii | 35-40 |
| Poloxamer | iii | 5 |
| Aristoflex | iv | 3-4 |
| KOH | vi | □ 1 |
| Etidronic acid | vi | □ 1 |
| EDTA (potassium) | vii | □ 1 |
| Sodium stannate trihydrate | vi | □ 1 |

TABLE 4
Stability of example 7

| | T | | | | Viscosity | Peroxide |
|---|---|---|---|---|---|---|
| | (° C.) | | month | pH | Pa · s | % |
| Tooth Whitening gel | <10° | C. | 0 | 6.3 | 37.4 | 10.1 |
| 10% H₂O₂ | | | 1 | 6.3 | 36.2 | 9.9 |
| | | | 2 | 5.6 | 45.0 | 10.2 |
| | | | 6 | 6.2 | 41.3 | 10.6 |
| | | | 12 | 6.1 | 37.6 | 9.9 |
| Tooth Whitening gel | 20-25° | C. | 0 | 6.3 | 37.4 | 10.1 |
| 10% H₂O₂ | | | 2 | 6.4 | 37.8 | 10.7 |
| | | | 3 | 6.0 | 35.5 | 10.6 |
| | | | 7 | 6.0 | 38.7 | 10.3 |
| | | | 13 | 5.9 | 38.7 | 9.9 |
| Tooth Whitening gel | 37° | C. | 0 | 6.3 | 37.4 | 10.1 |

| | | | | |
|---|---|---|---|---|
| 10% H₂O₂ | 2 | 6.0 | 37.0 | 9.8 |
| | 3 | 5.3 | 40.0 | 10.0 |

The composition according to the invention maintained its pH in the temperature range of from below 10° C. to 25° C. in the small range of above pH 5 and in particular below 7, preferred below 6.5. In addition the viscosity remained in the range of 10 to 100 Pa·s and was stable possessing values in the range of 20 to 45 Pa·s. Also the peroxide level remained in the range between 9 to 11% for at least 12 month in the temperature range of from below 10° C. to 25° C. At accelerated temperature of about 37° C. the peroxide level remained above 9% for 7 month analogous result were obtained with a composition of example 5.

1. A composition comprising:
i 2 wt. % to 30 wt. % of hydrogen peroxide;
ii water;
iii 1 wt. % to 10 wt. % poly(ethylene oxide)-poly(propylene oxide) block copolymer;
iv 0.01 wt. % to 10 wt. % of a salt of a polymer of acryolyl-2-alkyltaurate, acryolyl-2,2-dialkyltaurate, acryolyl-2-alkyltaurate salt or acryolyl-2,2-dialkyltaurate salt, or a mixture of at least two of the aforementioned taurates or selected from co-polymers of acryolyl-2-alkyltaurate, acryolyl-2,2-dialkyltaurate, acryolyl-2-alkyltaurate salt or acryolyl-2,2-dialkyltaurate salt or a mixture of at least two of the aforementioned taurates and at least one ethylenically unsaturated monomer or a mixture thereof, wherein the alkyl is selected from methyl, ethyl, n-propyl, iso-propyl, n-butyl, iso-butyl, tert-butyl groups, and wherein the salt is selected from ammonium, alkali, earth alkali or zinc salts of these polymers or co-polymers;
v 30 wt. % to 95 wt. % poly(ethylene oxide) homopolymer;
vi 0.01 wt. % to 5 wt. % stabilizer system; and
vii 0.01 wt. % to 1 wt. % of a chelating agent or a mixture of chelating agents;
wherein the composition comprises in total 100 wt. %.

2. Composition according to claim 1, wherein the pH of the composition is in the range of 5.0 to 7.5.

3. Composition according to claim 1, comprising:
i 5 wt. % to 15 wt. % of hydrogen peroxide;
ii 5 wt. % to 15 wt. % of water;
iii 2 wt. % to 10 wt. % poly(ethylene oxide)-poly(propylene oxide) block copolymer;
iv 0.01 wt. % to 10 wt. % salt of co-polymers selected from co-polymers of acryolyl-2-alkyltaurate, acryolyl-2,2-dialkyltaurate, acryolyl-2-alkyltaurate salt or acryolyl-2,2-dialkyltaurate salt or a mixture of at least two of the aforementioned taurates and at least one ethylenically unsaturated monomer, wherein the monomer is selected from vinylpyrrolidone, vinylpyrrolidone derivatives, wherein the alkyl is selected from methyl, ethyl, n-propyl, iso-propyl, n-butyl, iso-butyl, tert-butyl groups, and wherein the salt is selected from ammonium, alkali, earth alkali or zinc salts of these polymers or co-polymers;
v 60 wt. % to 95 wt. % poly(ethylene oxide) homopolymer;
vi 0.01 wt. % to 5 wt. % stabilizer system; and
vii 0.01 wt. % to 1 wt. % of a chelating agent or a mixture of chelating agents;
wherein the composition comprises in total 100 wt. %.

4. Composition according to claim 1, comprising:
i 15 wt. % to 30 wt. % of hydrogen peroxide;
ii 15 wt. % to 30 wt. % of water;
iii 2 wt. % to 10 wt. % poly(ethylene oxide)-poly(propylene oxide) block copolymer;
iv 0.01 wt. % to 10 wt. % salt of co-polymers selected from co-polymers of acryolyl-2-alkyltaurate, acryolyl-2,2-dialkyltaurate, acryolyl-2-alkyltaurate salt or acryolyl-2,2-dialkyltaurate salt or a mixture of at least two of the aforementioned taurates and at least one ethylenically unsaturated monomer, wherein the monomer is selected from vinylpyrrolidone, vinylpyrrolidone derivatives, wherein the alkyl is selected from methyl, ethyl, n-propyl, iso-propyl, n-butyl, iso-butyl, tert-butyl groups, and wherein the salt is selected from ammonium, alkali, earth alkali or zinc salts of these polymers or co-polymers;
v 30 wt. % to 60 wt. % poly(ethylene oxide) homopolymer;
vi 0.01 wt. % to 5 wt. % stabilizer system; and
vii 0.01 wt. % to 1 wt. % of a chelating agent or a mixture of chelating agents, wherein the composition comprises in total 100 wt. %.

5. Composition according to claim 1, wherein the weight content (wt. %) of hydrogen peroxide and water are from 2:1 to 1:2, optionally 1:1+/−10 wt. %.

6. Composition according to claim 1, wherein
iv the co-polymers are selected from co-polymers of acryolyl-2-alkyltaurate, acryolyl-2,2-dialkyltaurate, acryolyl-2-alkyltaurate salt or acryolyl-2,2-dialkyltaurate salt or a mixture of at least two of the aforementioned taurates and vinylpyrrolidone, wherein the alkyl is selected from methyl, and wherein the salt is selected from ammonium, alkali, earth alkali or zinc salts of these polymers or co-polymers.

7. Composition according to claim 1, wherein
iv the co-polymers are copolymers of an ammonium or alkali salt of acryloyldimethyltaurate and ethylenically unsaturated monomer, acryloyldimethyltaurate and styrene or acryloyldimethyltaurate and vinylpyrrolidone.

8. Composition according to claim 1, wherein
iv the co-polymers are selected from ammonium acryloyldimethyltaurate and vinylpyrrolidone copolymer.

9. Composition according to claim 1, wherein
vii the chelating agent or the mixture of chelating agents is selected from ethylenediaminetetraacetic acid, alkali and/or alkali earth salt of ethylenediaminetetraacetic acid (EDTA), aromatic chelating agent and a mixture thereof.

10. Composition according to claim 1, wherein
vi the stabilizer system is selected from
a) phosphorus containing stabilizer or mixture of phosphorus containing stabilizers selected from the group consisting of pyrophosphate, diethylene triamine penta(methylene phosphonic acid), hexamethylene diamine tetra(methylene phosphonic acid), bis hexamethylene triamine pentamethylene phosphonic acid and etidronic acid, and
b) stannate stabilizer, and/or
c) alkali hydroxide, base and/or buffers.

11. Composition according to claim 1, wherein
vi the stabilizer system is selected from a) etidronic acid and b) stannate stabilizer, optionally sodium stannate trihydrate.

12. Composition according to claim 1, wherein the composition comprises
v 35 wt. % to 85 wt. % of poly(ethylene oxide) homopolymer.

13. Composition according to claim 1, wherein
v poly(ethylene oxide) homopolymer comprises terminal hydroxyl groups.

14. Composition according to claim 1, wherein the composition comprises
iv 0.5 wt. % to 5 wt. % of an ammonium or alkali salt of a copolymer of acryloyldimethyltaurate and vinylpyrrolidone.

15. Composition according to claim 1, wherein
iii is a poly(ethylene oxide)-poly(propylene oxide)-poly(ethylene oxide) block copolymer, optionally with terminal hydroxyl groups, and optionally with a medium molecular weight of 9840 to 14600 Dalton (Dalton D=unit for the relative molecular mass).

16. A method of encapsulating a mixture comprising at least hydrogen peroxide, water and poly(ethylene oxide) homopolymer, or for forming a matrix comprising a mixture of at least hydrogen peroxide, water and poly(ethylene oxide) homopolymer, said method comprising adding to said mixture a salt of co-polymers selected from co-polymer of acryolyl-2-alkyltaurate, acryolyl-2,2-dialkyltaurate, acryolyl-2-alkyltaurate salt or acryolyl-2,2-dialkyltaurate salt or a mixture of at least two of the aforementioned taurates and at least one ethylenically unsaturated monomer or a mixture thereof, wherein the alkyl is selected from methyl, ethyl, n-propyl, iso-propyl, n-butyl, iso-butyl, tert-butyl groups, and wherein the salt is selected from ammonium, alkali, earth alkali or zinc salts of these co-polymers.

17. Method of oral care comprising applying to teeth the composition according to claim 1.
